(19) United States
(12) Patent Application Publication
(10) Pub. No.: US 2013/0245763 A1
(43) Pub. Date: Sep. 19, 2013

(54) THREADED IMPLANT

(71) Applicant: Richard G. Mauldin, Erie, CO (US)

(72) Inventor: Richard G. Mauldin, Erie, CO (US)

(21) Appl. No.: 13/791,801

(22) Filed: Mar. 8, 2013

Related U.S. Application Data
(60) Provisional application No. 61/609,211, filed on Mar. 9, 2012.

(51) Int. Cl. A61F 2/44

(52) U.S. Cl. CPC A61F 2/4455
USPC 623/17.11

(57) ABSTRACT

A threaded implant includes a body, exterior surface, interior surface, fenestrations, and threads. The body may be a hollow tubular structure. Alternatively, the body may be solid and cannulated. The surface of the threaded implant may have mesh configuration, beaded configuration, trabecular configuration, holes or any surface conducive to bony in-growth, on-growth and/or through-growth. The threads may be barbed and may allow for easy insertion through the bone. The threaded implant may be tapped into place. A method for fusing bone may involve inserting the implant laterally through the ilium, through the sacral-iliac joint, and into the sacrum.

### CROSS REFERENCE TO RELATED APPLICATIONS

This patent application claims priority to U.S. Provisional Application No. 61/609,211, titled “THREADED IMPLANT”, filed on Mar. 9, 2012. This patent application may be related to one or more of the following patent applications: U.S. Patent Publication No. 2011/0087294, titled “SYSTEMS AND METHODS FOR THE FUSION OF THE SACRAL-ILIAC JOINT”, filed on Oct. 5, 2010 and U.S. Patent Publication No. 2011/0118785, titled “APPARATUS, SYSTEMS, AND METHODS FOR ACHIEVING ANTERIOR LUMBAR INTERBODY FUSION”, filed on Dec. 6, 2010. Each of these references is herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

The present invention generally relates to the fixation or fusion of the sacroiliac joint.

### BACKGROUND

Many types of hardware are available both for the fixation of bones that are fractured and for the fixation of bones that are to be fused. A fusion is an operation where two bones, usually separated by a joint, are allowed to grow together into one bone. The medical term for this type of fusion procedure is arthrodesis.

For example, the human hip girdle (see FIGS. 4 and 5) is made up of three large bones joined by two relatively immobile joints. One of the bones is called the sacrum and it lies at the bottom of the lumbar spine, where it connects with the L5 vertebra. The other two bones are commonly called “hip bones” and are technically referred to as the right ilium and the left ilium. The sacrum connects with both hip bones at the left and right sacroiliac joint (in shorthand, the SI-Joint).

The SI-Joint functions in the transmission of forces from the spine to the lower extremities, and vice-versa. The SI-Joint has been described as a pain generator for up to 22% of lower back pain.

To relieve pain generated from the SI-Joint, sacroiliac joint fusion is typically indicated as a surgical treatment, e.g., for degenerative sacroiliitis, inflammatory sacroiliitis, iatrogenic instability of the sacroiliac joint, osteitis condensans ilii, or traumatic fracture dislocation of the pelvis. Currently, screws and screws with plates are used for sacro-iliac fusion. At the same time the cartilage has to be removed from the “synovial joint” portion of the SI-joint. This requires a large incision to approach the damaged, subluxed, dislocated, fractured, or degenerative joint.

There is a need for improved SI-Joint bone fusion treatments for addressing chronic hip, joint or back pain.

### SUMMARY OF THE DISCLOSURE

The present invention generally relates to the fixation or fusion of the sacroiliac bone joint.

Some embodiments provide for a threaded bone implant with an implant body having a distal end and a proximal end, and a slot located at the proximal end of the body. The implant may have a length between about 30 mm to about 70 mm. Additionally, the implant body can include a wall with an exterior surface, an interior surface, and a thickness through the wall between the surfaces. In further variations, the slot is configured to receive a driving device.

In various embodiments, the implant body has a first diameter at the distal end and a second diameter at the proximal end, the second diameter being greater in dimension that the first diameter. The first and second diameters may be between about 5 mm and about 15 mm. In additional embodiments, the implant body is tapered along a longitudinal direction between the distal and proximal ends. The threaded implant can also be configured to be inserted laterally into sacral-iliac joint between an iliac and a sacrum.

The implant body may include a plurality of pores on the body, wherein each pore of the plurality of pores does not extend through the entire thickness of the wall between the interior and exterior surfaces. In any of the preceding embodiments, each pore of the plurality of pores can be configured to not penetrate through the entire thickness of the wall. In further variations, the pores or plurality of pores may have a mean pore size between about 50 to 800 microns.

In any of the preceding embodiments, the implant may be threaded. The implant body may comprise a plurality of threads on an exterior surface of the body. The threads may also comprise proximally directed barbs adapted to facilitate driving the implant into bone and to inhibit migration out of bone.

In any of the preceding embodiments, the implant can be made from a bioabsorpable material such that the implant is bioapsorbable.

In any of the preceding embodiments, the implant may include a safety protrusion or safety marking to indicate implant insertion depth.

Other embodiments provide for a threaded bone implant including a hollow body with a distal end and a proximal end. The hollow body may be covered with a plurality of pores where the pores may have a mean pore size between about 50 microns to about 800 microns. Each of the pores may have a mean pore size between about 50 microns to about 800 microns. In some variations, each of the pores does not does not penetrate through the entire thickness of the wall. The implant may also include a head located at the proximal end of the body. In some cases, the head includes a head slot. In other variations, the head comprises a larger cross-section relative to the body.

The implant may also be configured for insertion into adjacent bone segments.

In any of the preceding embodiments, the implant or implant body may be formed from a trabecular porous metal. In other variations, the implant or implant body is formed from a solid metal coated with porous metal.

In any of the preceding embodiments, the implant body includes a threaded portion. In other embodiments, the threaded portion of the implant body may be configured to cut a helical groove in bone during insertion. Moreover, the threaded portion may include a pitch configured to stabilize the adjacent bone segments. In any of the preceding embodiments, the pitch may be between 0.5 mm to about 4 mm. The threaded portion may also be tapered near the distal end of the body. In other embodiments, the implant body may include a smooth section as well as a threaded section or portion.

In any of the preceding embodiments, the implant may include a cannula extending through head of the implant and between the distal and proximal ends of the implant body. In some variations, the cannula is configured to receive a guide pin.

In any of the preceding embodiments, the implant may be formed from a material conducive to bony in-growth, on-growth or through-growth. The implant body may include an outer layer having a surface feature promoting bony in-growth. In some embodiments, surface feature may have fenestrations. In other embodiments, the surface feature is a porous mesh. In further embodiments, the surface feature includes a porous plasma spray coating. In some cases, the surface feature includes a surface coating with a biologic aid for promoting bony in-growth. In any of the preceding embodiments, the biologic aid comprises growth factors. Additionally, in any of the preceding embodiments, the biologic aid is a controlled release formulation.

Further embodiments provide methods for relieving joint pain. These methods include the steps of identifying a joint having a first bone segment, a second bone segment, and a non-bony region between the first and second bone segments; providing a threaded bone fusion implant with a body with a threaded outer surface; inserting a delivery pin through a first bone segment, through the non-bony region, and into a second bone segment; forming a pilot bore in the first and second bone segments and through the non-bony region; and inserting the threaded implant into the first and second bone segments to thereby fuse the bone segments.

In any of the preceding methods, the implant body may include a wall covered with a plurality of pores, wherein the plurality of pores has a mean pore size between about 50 microns to about 800 microns. Additionally, the implant body may be formed from a trabecular porous metal or a solid metal coated with porous metal. In some embodiments, the implant body includes a wall with an exterior surface, an interior surface, and a thickness through the wall between the surfaces. The implant body may also include a plurality of pores, where each pore of the plurality of pores does not extend through the entire thickness of the wall between the interior and exterior surfaces.

Additionally, in any of the preceding embodiments, inserting the threaded implant further includes screwing the implant into the pilot bore. In other embodiments, inserting the threaded implant further includes tapping the implant into the pilot bore and positioning the implant in the first segment, through the non-bony region, and partially through the second bone segment. Forming the pilot bore may include shaping the pilot bore to have a tapered shape corresponding to a tapered section of the implant body.

Other embodiments provide methods for fusion of the sacral-iliac joint between an ilium and a sacrum. These methods may include inserting a threaded implant laterally near the ilium, the implant including a proximal end and a distal end; and driving the distal end of threaded implant into the ilium, through the sacral-iliac joint, and into the sacrum.

In any of the preceding methods, the implant may include a body having a wall covered with a plurality of pores, wherein the plurality of pores has a mean pore size between about 50 microns to about 800 microns. The body may be formed from a trabecular porous metal. Additionally, the body may be formed from a solid metal coated with porous metal. In some cases, the body comprises a wall with an exterior surface, an interior surface, and a thickness through the wall between the surfaces and each pore of the plurality of pores does not extend through the entire thickness of the wall between the interior and exterior surfaces.

In any of the preceding embodiments, inserting the threaded implant further includes screwing the implant through the ilium, through the sacral-iliac joint, and partially through the sacrum. In other embodiments, the methods further include positioning the threaded implant by directly tapping the implant into through the sacral-iliac joint without forming a pilot bore. In other embodiments, driving the distal end of the threaded implant includes engaging a slot on proximal end of the threaded implant with a driving device.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1A is a perspective view of an exemplary threaded implant.

FIG. 1B is a longitudinal sectional view of the exemplary threaded implant of FIG. 1A.

FIG. 1C is a side view of the exemplary threaded implant of FIG. 1A

FIG. 2A is a side view of another exemplary threaded implant.

FIG. 2B is an enlarged view of the distal end of the exemplary implant of FIG. 2A.

FIGS. 3A-3D are illustrations of an exemplary procedure for implanting a threaded implant.

FIGS. 4-5 are, respectively, anterior and posterior anterior views of the human hip girdle including the sacrum and the hip bones (the right ilium, and the left ilium), the sacrum being connected with both hip bones at the sacroiliac joint (SI-Joint).

FIGS. 6-8B are anatomic views showing, respectively, in pre-implanted perspective, implanted perspective, implanted anterior view, and implanted cranio-caudal section view, the implantation of three implant structures for the fixation of the SI-Joint using a lateral approach through the ilium, the SI-Joint, and into the sacrum.

FIGS. 9A-9B show a perspective view and cross-sectional view of a porous threaded implant.

FIGS. 10A-10B show a side view and cross-sectional view of another exemplary porous threaded implant.

### DETAILED DESCRIPTION

Reference will now be made in detail to exemplary embodiments of the invention, examples of which are illustrated in the accompanying drawings. While the invention will be described in conjunction with the exemplary embodiments, it will be understood that they are not intended to limit the invention to those embodiments. On the contrary, the invention is intended to cover alternatives, modifications and equivalents, which may be included within the spirit and scope of the invention as described herein.

Various aspects of the present invention relate to a threaded implant having fenestrations for bony in-growth, on-growth and/or through growth. In various embodiments, the threaded implants may be used to fuse the sacroiliac joint.

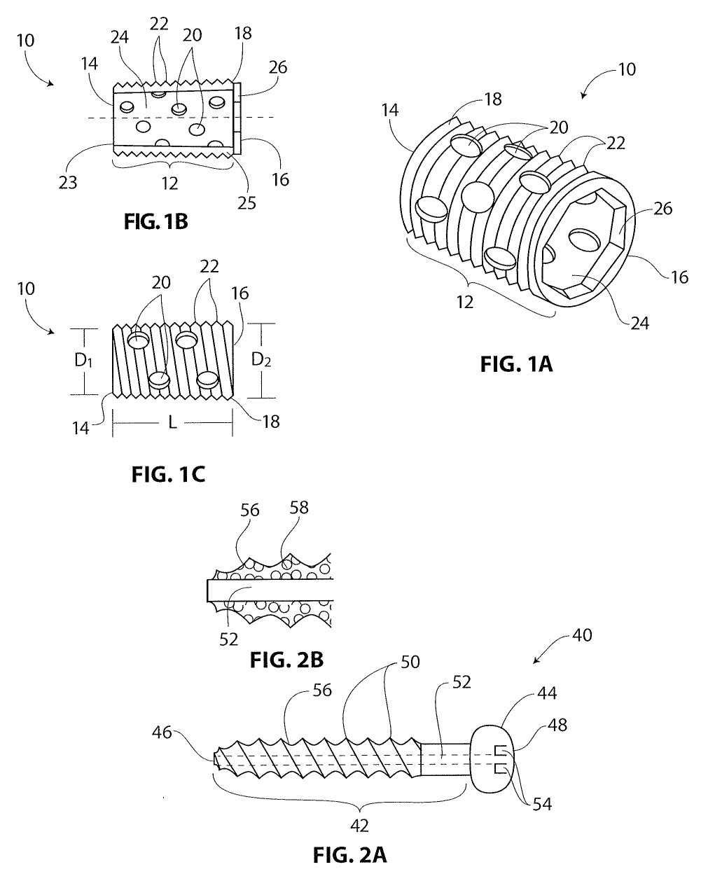

FIG. 1A is a perspective view of an exemplary threaded implant. Threaded implant 10 may include a body 12 having a distal end 14 and a proximal end 16, where the terms distal and proximal are used in relation to the physician inserting the threaded implant 10. Body 12 includes an exterior surface 18, fenestrations 20, threads 22 and an interior surface 24. In various embodiments, the body 12 is a hollow tubular structure that includes threads 22 on the exterior surface 18. The threads 22 on the exterior surface 18 may allow for easy insertion through the bone. In various embodiments, threads 22 are barbed, pointing toward the proximal end of the implant. This arrangement facilitates driving the implant into bone, and inhibits migration back out of the bone. The interior of the proximal end 16 may include a slot 26. Slot 26 may form a hollow geometrical shape, such as a hexagon, octagon or other geometrical shape, into which an inserted part is designed to fit. In various embodiments, the slot 26 is designed to fit and receive a driving device.

Threaded implant 10 may be formed from a durable material usable in the prosthetic arts that is not subject to significant bio-absorption or resorption by surrounding bone or tissue over time. Threaded implant 10 is intended to remain in place for a time sufficient to stabilize the fracture or fusion site. Threaded implant 10 may also remain in place in the patient permanently. Such materials include, but are not limited to, titanium, titanium alloys, tantalum, tivanium (aluminum, vanadium, and titanium), chrome cobalt, surgical steel, or any other total joint replacement metal and/or ceramic, sintered glass, artificial bone, any uncemented metal or ceramic surface, or a combination thereof. Alternatively, the threaded implant 10 may be formed from a suitable durable biologic material or a combination of metal and biologic material, such as a biocompatible bone-filling material or bone. The threaded implant 10 may be molded from a flowable biologic material, e.g., acrylic bone cement, that is cured, e.g., by UV light, to a non-flowable or solid material, e.g. polymers such as PLA, PLGA, PGA, or other similar materials. Threaded implant 10 may have a portion on the outer surface that is conducive to bony in-growth, on-growth and/or through-growth. In various embodiments, the portion may include the entire surface, including exterior surface 18 and interior surface 24, of the threaded implant 10. The bony in-growth, on-growth and/or through-growth portion may include through holes, various surface patterns, various surface textures, and/or pores, or combinations thereof. For example, the exemplary embodiment illustrated in FIG. 1A includes a plurality of fenestrations 20. The exterior surface 18 may have a mesh configuration, beaded configuration, trabecular configuration, holes or any surface conducive to bony in-growth, on-growth and/or through-growth.

The exterior surface 18 and interior surface 24 of the threaded implant 10 may be coated, wrapped or surface treated to promote the bony in-growth, on-growth and/or through-growth. In various embodiments, the coating material can include a biologic aid that can promote and/or enhance bony ingrowth, tissue repair, and/or reduce inflammation, infection and pain. The biologic aid may include growth factors, such as bone morphogenetic proteins (BMPs), hydroxyapatite in a liquid or slurry carrier, demineralized bone, morselized autograft or allograft bone, medications to reduce inflammation, infection and pain such as analgesics, antibiotics and steroids. In various embodiments, the growth factors may be human recombinant growth factors, such as hr-BMP-2 and/or hr-BMP-7, or any other human recombinant form of BMP.

The carrier for the biologic aid may be a liquid or gel such as saline or a collagen gel. The biologic aid may also be encapsulated or incorporated in a controlled released formulation so that the biologic aid is released to the patient at the implant site over a longer duration. For example, the controlled release formulation may be configured to release the biologic aid over the course of days, weeks or months, and can be configured to release the biologic aid over an estimated time it would take for the implant site to heal. The amount of biologic aid delivered to the threaded implant 10 may be controlled using a variety of techniques, such as controlling or varying the amount of coating material applied to the threaded implant 10 and/or controlling or varying the amount of biologic aid incorporated into the coating material. Controlling the amount of biologic aid delivered may be important because excessive use of certain biologic aids may result in negative effects such as localized inflammation, local pain or radicular pain.

In a various embodiments, the bony in-growth portion, on-growth and/or through-growth portion includes a porous plasma spray coating on the threaded implant 10. In various embodiments, the entire surface of threaded implant 10, including exterior surface 18 and interior surface 24, includes a porous plasma spray coating. The coating may create a biomechanically rigorous fixation/fusion system, designed to support reliable fixation/fusion and acute weight bearing capacity.

The threaded implant 10 may be formed from a material that itself inherently possesses a structure conducive to bony in-growth, on-growth and/or through-growth, such as a porous mesh, hydroxyapatite, or other porous surface. In various embodiments, the threaded implant 10 may be formed from a porous metal, such as trabecular metal, for bony in-growth, on-growth and/or through-growth. The bony in-growth, on-growth and/or through-growth portion may further be covered with various other coatings such as antimicrobial, antithrombotic, and osteoinductive agents, or a combination thereof. In various embodiments, the entire threaded implant 10 may be impregnated with such agents.

Implant stability may be defined as the ability of the implant to resist loads in the axial, lateral and rotational directions without loosening. The ability of the implant to withstand these loads while maintaining the stability is important. Primary implant stability is achieved at the time of surgery and may depend on the implant design. Primary implant stability may be influenced by implant geometry. While the initial stability may be related to mechanical features, the bone healing process eventually dictates long-term stability.

Secondary implant stability, which is achieved over time, may depend on the level of primary stability and the biological response to the surgery and implant. Newly formed bone tissue may fill voids and openings, such as fenestrations 20, at the implant/bone interface, create direct contact with the implant surface, and engage with surface irregularities. This interlocking effect may be amplified when newly formed bone matures over time and may provide stability against rotation, migration and micromotion once implanted.

Threaded implant 10 may include a safety feature for preventing the implant from being driven too far into a patient. In various embodiments, the safety feature may include a marking, a protrusion, or some other feature. The protrusion may be located on implant 10 or the delivery device (not shown) and may come in contact with a patient's skin or outer ilium surface to prevent further advancement into the bone. The marking may be located on implant 10 or on the delivery device and may indicate a measure of the insertion depth, for example a depth gauge.

FIG. 1B is a longitudinal sectional view of the exemplary threaded implant of FIG. 1A. Threaded implant 10 includes body 12, distal end 14, proximal end 16, exterior surface 18, interior surface 24, fenestrations 20, threads 22 and slot 26. As shown the longitudinal section view, the body is monolithically formed. In various embodiments, the body 12 may be formed as two or more pieces. The two or more pieces may include an inner core 23 and an outer layer 25. The outer layer 25 may be threaded and may be porous to promote bony in-growth, on-growth and/or through growth. Fenestrations 20 may extend through both the inner core 23 and outer layer 25.

FIG. 1C is a side view of the exemplary threaded implant of FIG. 1A. The implant of FIG. 1C has a length L, distal end diameter D₁ and proximal end diameter D₂. The threaded implant 10 may be sized according to the local anatomy. The morphology of the local structures can be generally understood by medical professionals using textbooks of human skeletal anatomy along with their knowledge of the site and its disease or injury. The physician is also able to ascertain the dimensions of the threaded implant 10 based upon prior analysis of the morphology of the targeted bone region using, for example, plain film x-ray, fluoroscopic x-ray, or MRI or CT scanning. In various embodiments, the length L of the threaded implant 10 is in the range of about 30 mm to 70 mm. In various embodiments, the length L of the threaded implant 10 is about 30 mm, 35 mm, 40 mm, 45 mm, 50 mm, 55 mm, 60 mm, 65 mm, or 70 mm.

The dimension of diameters D₁ and D₂ may be equal or not equal. When D₁ and D₂ are equal, body 12 may have a uniform shape in the longitudinal direction. When D₁ and D₂ are not equal, body 12 may have a tapered shape. In various embodiments, D₂ may be greater in dimension than D₁ to form an inwardly tapered shape from the proximal end 16 to the distal end 14 of body 12, as shown. In various embodiments, the dimensions of D₁ and D₂ are in the range of between about 5 mm to about 15 mm. In various embodiments, the dimensions of D₁ and D₂ are about 5 mm, 7 mm, 9 mm, 11 mm, 13 mm or 15 mm.

FIG. 2A is a side view of another exemplary threaded implant. Threaded implant 40 may include body 42, head 44, distal end 46 and proximal end 48. Body 42 may include threads 50 and cannula 52. Threaded implant 40 of FIG. 2A may be formed from the same materials as implant 10 of FIG. 1A. Threaded implant 40 of FIG. 2A may be constructed or fabricated similarly to implant 10 of FIG. 1A. In various embodiments, a guide pin (not shown) may be inserted through cannula 52 to secure and guide threaded implant 40. Head 44 may include one or more head slots 54. Head slot 54 may be designed to fit and receive a driving device. In various embodiments, the driving device is a manual hex driver or powered driver such as a drill. Head 44 may have a larger cross-section than body 42 to control the distance threaded implant 40 may be driven into bone. Threaded implant 40 may also include safety features previously described with respect to threaded implant 10 of FIG. 1A.

An exterior surface 56 may cover body 42 and head 44. The exterior surface 56 may have a coating (See FIG. 2B). The coating surrounding exterior surface 56 may be similar to that described with respect to threaded implant 10 of FIG. 1A.

Threads 50 may extend over a portion of body 42. The threaded portion of body 42 may include a portion at the distal end that is tapered. In various embodiments, a portion of body 42 is smooth. The pitch of threads 50 may be configured to cut a helical groove in bone as implant 42 is inserted. In various embodiments, the pitch of threads 50 is configured to stabilize or fuse the SI-Joint. The pitch of threads 50 may be selected based on the material composition of the threaded implant 40. The pitch of threads 50 may be configured to allow implantation from a lateral approach. In various embodiments, the pitch of the threads 50 is in the range of about 0.5 mm to 4 mm.

FIG. 2B is an enlarged view of the distal end of the exemplary implant of FIG. 2A. As illustrated in FIG. 2B, implant 40 is made of a porous material and the exterior surface 56 may include a porous coating. In various embodiments, the porous coating may be a plasma spray coating as described with respect to FIG. 1A. The porous coating includes a plurality of pores 58 which may promote bony in-growth, on-growth and/or through growth. In various embodiments, the mean pore size of the porous material is in the range of about 50 microns to about 800 microns.

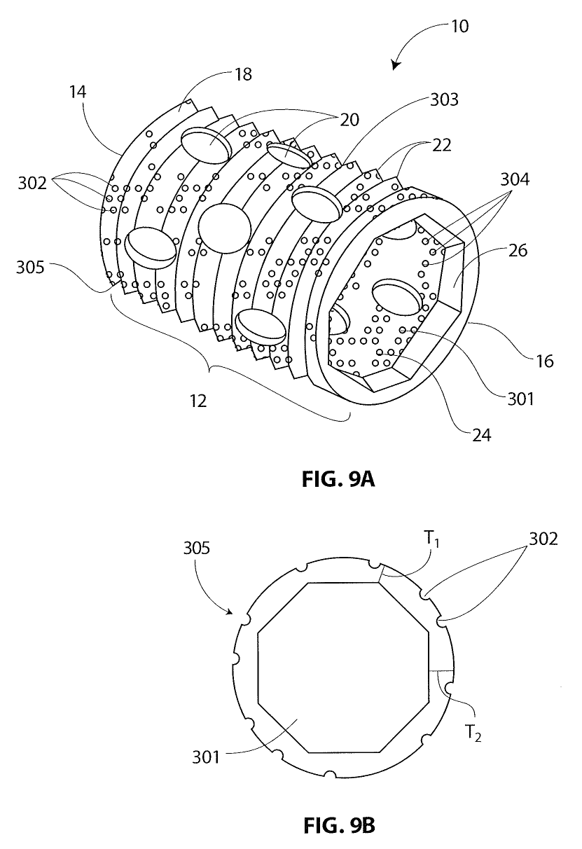

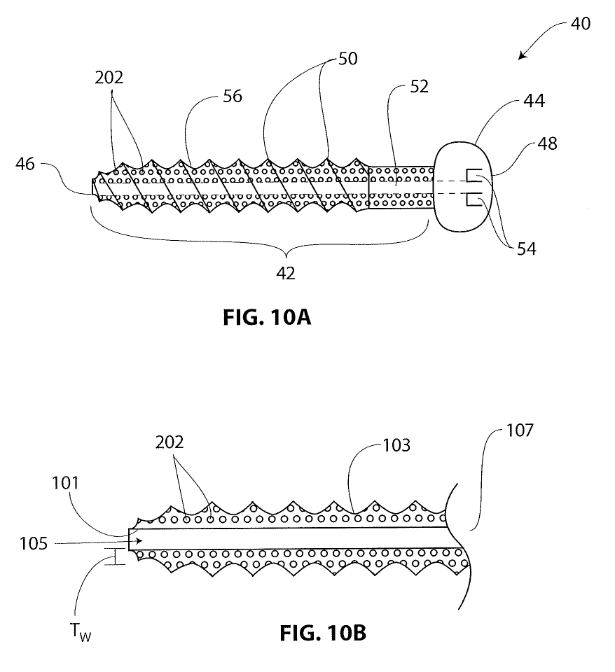

FIGS. 9A-10B show additional details regarding the plurality of pores that may cover all or a portion of the implant. FIG. 9A shows an implant 10′ similar to implant 10 of FIG. 1A-1C. As shown, the body of the implant is formed by a wall 303 having an inner side or surface 301 and an outer side/surface 305. A series of pores 302 are located on the wall on the outer surface. In various embodiments, each of the pores on the wall does not penetrate directly through the entire wall thickness T_{W}. Rather, the depth of the pore is less than the full thickness of the wall at which the pore is positioned. For example, FIG. 9B shows a cross-section of the implant 10 having two different wall thicknesses, T₁ (at an apex of the slot) and T₂ (at a midpoint between slot apexes), at different sections of the wall. In either case, pores located near at those wall sections do not directly penetrate through the entire wall. Moreover, the depth of the pores are less than the wall thickness. As such, the pores do not form holes or openings that directly extend from the outside surface 305 into the interior surface 301. In some embodiments, the implant may be formed of a porous material having a matrix of channels that indirectly connect inner surface 301 with outer surface 305, but do not directly connect the two surfaces as do fenestrations 20.

In addition to the pores on the outside surface 305, a plurality of pores 304 may be located on the inside surface 301. These pores may be formed in the same manner described and also do not entirely penetrate through the wall 303.

FIGS. 10A-B show a plurality of pores 202 on the implant 40 of FIGS. 2A-B. As shown in this embodiment, the pores 202 also do not directly penetrate through the full thickness of the wall 107. Implant 40 is shown having a wall 107 that defines a lumen 105 within the implant 40. The lumen is surrounded by an inner surface 101 that may also include pores. These pores do not directly extend through the full thickness of the wall. Likewise, the outer surface 103 can include a plurality of pores that have a depth of penetration less than the full thickness of the wall.

In some cases, the entire body of the implant may be covered by pores as a porous material, such as a trabecular porous metal forms the entire implant body. In other cases, the body is coated fully or partially with a porous coating such as a porous metal coating. In each case, the pores encourage rapid ingrowth of tissue into the implant to permit the implant to be fused into place within the surrounding bone segments. In some embodiments, fenestrations 20 are omitted, so that the implant promotes tissue ingrowth but not through-growth. In such embodiments, the implant may be rapidly fused with the bone but still removable if desired by applying sufficient torque to the implant to break the tissue ingrowth.

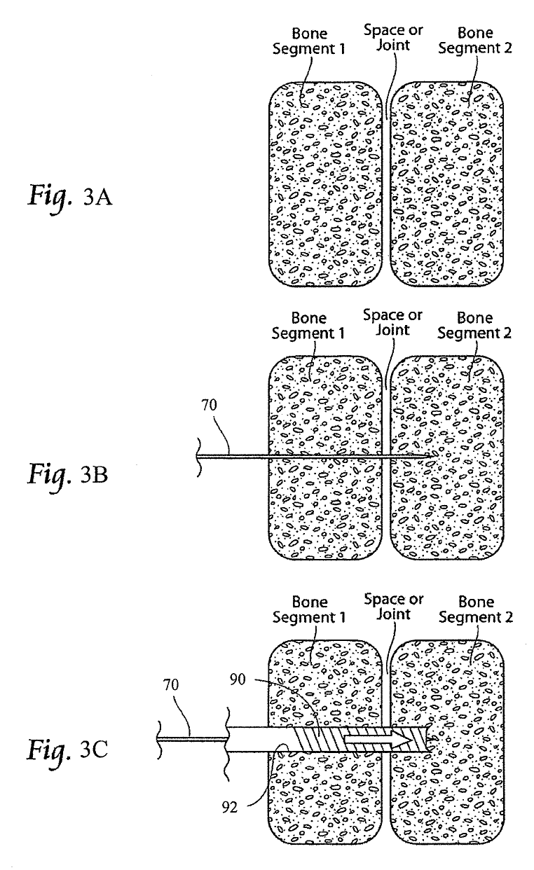

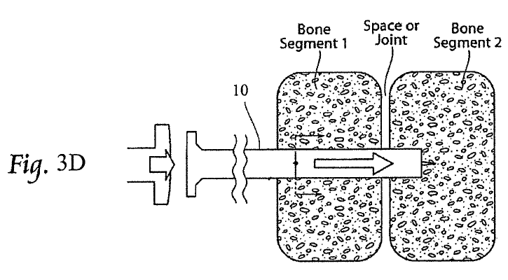

FIGS. 3A-3D are illustrations of an exemplary procedure for implanting the threaded implants of FIGS. 1A-2B. More detailed, anatomically-focused descriptions of particular implantation techniques of the threaded implants in the SI-Joint are described with respect FIGS. 4-8B.

The physician identifies the bone segments or adjacent bone regions that are to be fixated or fused (arthrodesed) (see FIG. 3A). Aided by conventional visualization techniques, e.g., using X-ray image intensifiers such as a C-arms or fluoroscopes to produce a live image feed that is displayed on a TV screen, a delivery pin 70 is introduced by conventional means (see FIG. 3B) through the one adjacent bone segment or region (bone segment 1), through the intervening space or joint, and partially into the other adjacent bone segment or region (bone segment 2).

A cannulated drill bit 90 may be passed over the delivery pin 70 (see FIG. 3C), to form a pilot insertion path or bore 92 through the one adjacent bone segment or region, through the intervening space or joint, and partially into the other adjacent bone segment or region. A single drill bit or multiple drill bits 90 may be employed to drill through bone fragments or bone surfaces to create a pilot bore 92 of the desired size and configuration. A tapered drill bit or other cutting tool may be used to create a pilot bore having a taper that is complementary to a taper of the implant. When the pilot bore 92 is completed, the cannulated drill bit 90 is removed.

A threaded implant 10 (see FIG. 3D) may be tapped over the delivery pin 70 through the pilot bore 92. The threaded implant 10 is advanced by tapping into the pilot bore 92 through the one adjacent bone segment or region, through the intervening space or joint, and partially into the other adjacent bone segment or region. Alternatively, threaded implant 10 may screwed into pilot bore 92 through the one adjacent bone segment or region, through the intervening space or joint, and partially into the other adjacent bone segment or region. In some embodiments utilizing a tapered implant and a tapered bore, a torque wrench may be utilized to indicate to the surgeon when the implant is sufficiently seated across the joint.

Threaded implant 10 may also be positioned without forming a pilot insertion path or bore 92. Threaded implant 10 may be positioned by directly tapping or screwing the delivery device until progress is prevented by the safety stop feature as described with respect to FIGS. 1A-2B. (See FIG. 3D)

Bone graft or bone graft substitute (not shown) may be packed into implant 10 and bore 92. The bone graft may be autologous, allograft, or synthetic. Substitute bone graft may include ceramics (i.e. calcium phosphate, hydroxyapatite, tricalcium phosphate), bioglass and calcium sulphate or other biologic aid. Biologic aids and carriers for biologic aids are described with respect to FIG. 1A. Threaded implant 10 and bore 92 may be treated with bone graft or bone graft substitute before implantation. In various embodiments, threaded implant 10 and bore 92 are treated with bone graft or bone graft substitute after implantation. The addition of bone graft or substitute bone graft may enhance secondary implant stability to the newly fused joint and may provide resistance to rotation, migration and micromotion.

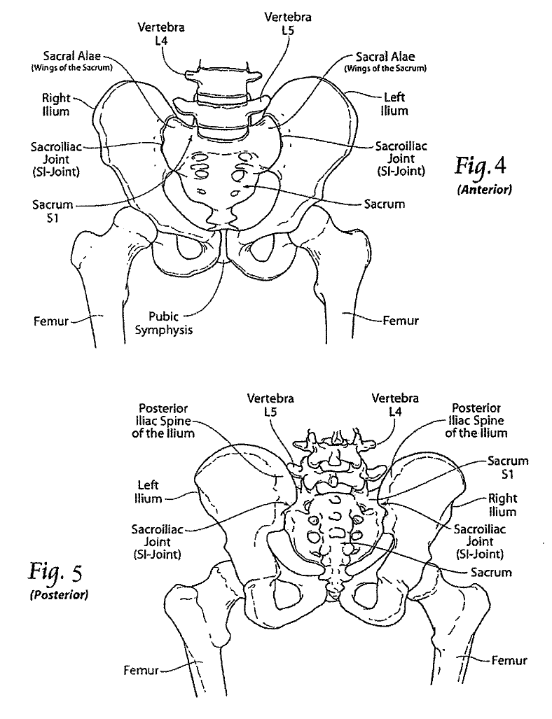

FIGS. 4-5 are, respectively, anterior and posterior anterior views of the human hip girdle including the sacrum and the hip bones (the right ilium, and the left ilium), the sacrum being connected with both hip bones at the sacroiliac joint (in shorthand, the SI-Joint). Threaded implant structures 10, 40 like that shown in FIGS. 1A and 2A make possible the fixation of the SI-Joint (shown in anterior and posterior views, respectively, in FIGS. 4 and 5) in a minimally invasive manner. Threaded implant structures 10, 40 may be effectively implanted through the use of two alternative surgical approaches; namely, a lateral approach or a postero-lateral approach. Either procedure is desirably aided by conventional lateral and/or anterior-posterior (A-P) visualization techniques, e.g., using X-ray image intensifiers such as a C-arms or fluoroscopes to produce a live image feed that is displayed on a TV screen. Although threaded implant 10 is illustrated in FIGS. 3D, 6-8B, threaded implant 40 may be implanted using the method described herein.

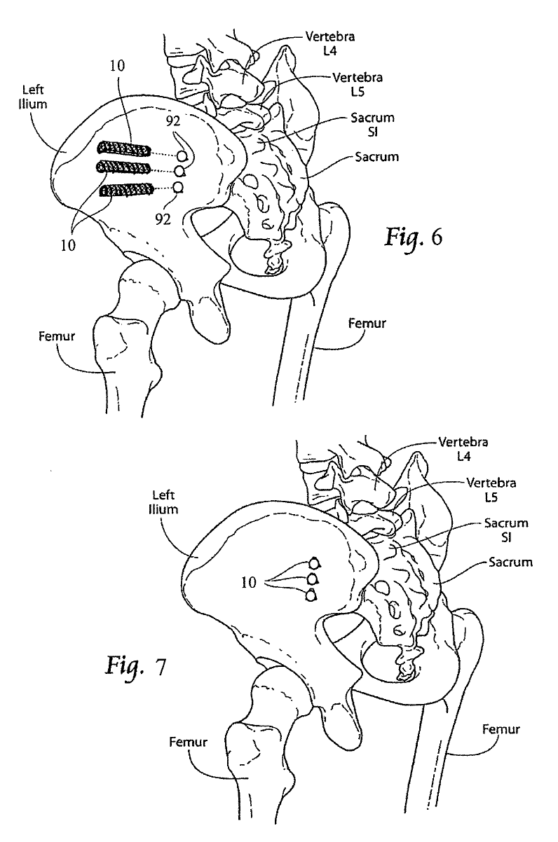

FIGS. 6-8B are anatomic views showing, respectively, in pre-implanted perspective, implanted perspective, implanted anterior view, and implanted cranio-caudal section view, the implantation of three implant structures for the fixation of the SI-Joint using a lateral approach. In one embodiment of a lateral approach (see FIGS. 6-8B), one or more threaded implant structures 10 are introduced laterally through the ilium, the SI-Joint, and into the sacrum. This path and resulting placement of the threaded implant structures 10 are best shown in FIGS. 6-8B. In the illustrated embodiment, three threaded implant structures 10 are placed in this manner. Also in the illustrated embodiment, the threaded implant structures 10 are circular in cross section, but it should be appreciated that threaded implant structures of other cross sections may be used.

Before undertaking a lateral implantation procedure, the physician identifies the SI-Joint segments that are to be fixated or fused (arthrodesed) using, e.g., the Fortin finger test, thigh thrust, FABER, Gaenslen's, compression, distraction, and diagnostic SI Joint injection.

In an exemplary procedure aided by lateral, inlet, and outlet C-arm views, and with the patient lying in a prone position, the physician aligns the greater sciatic notches and then the alae (using lateral visualization) to provide a true lateral position. A 3 cm incision is made starting aligned with the posterior cortex of the sacral canal, followed by blunt-tissue separation to the ilium.

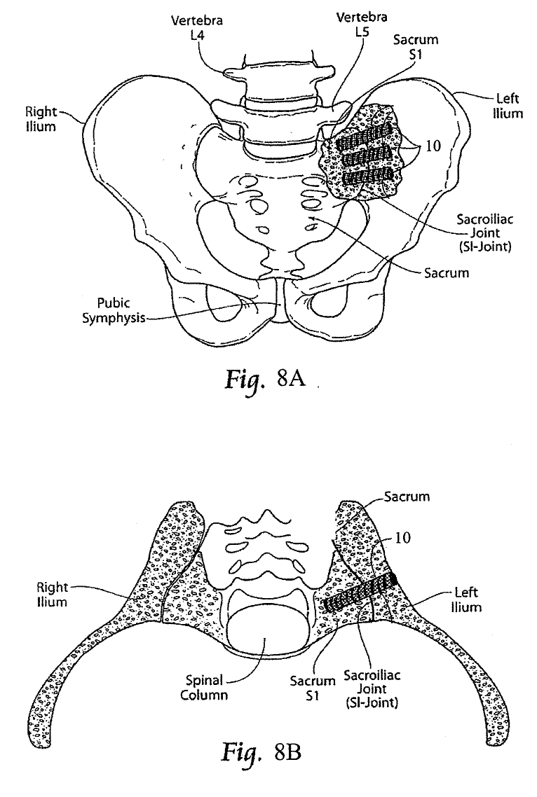

From the lateral view, the delivery pin 70, a Steinmann Pin for example, with a pin sleeve (not shown), is started resting on the ilium at a position inferior to the sacrum end plate and just anterior to the sacral canal and at a shallow angle (e.g. 15° to 20° off the floor, as FIG. 8B illustrates). In the outlet view, the delivery pin 70 should be parallel to the sacrum end plate or angled slightly away from the sacrum end plate. In a lateral view, the delivery pin should be posterior to the sacrum anterior wall. In the outlet view, the delivery pin should be superior to the first sacral foramen and lateral of mid-line. This corresponds generally to the sequence shown diagrammatically in FIGS. 3A-B. A soft tissue protector (not shown) is desirably slipped over the delivery pin 70 and firmly against the ilium before removing the pin sleeve. In the inlet view, the trajectory of the delivery pin 70 must not penetrate the anterior sacral cortex.

The pilot bore 92 may be drilled in the manner previously described over the delivery pin 70 (and through the soft tissue protector), as illustrated in FIG. 3C. The pilot bore 92 may extend through the ilium, through the SI-Joint, and into the sacrum. The drill bit 90 is then removed.

The threaded implant 10 is tapped into the pilot bore 92 over the delivery pin 70 (and through the soft tissue protector).

As shown in FIGS. 6 and 7, a tubular threaded implant 10 may be tapped through the soft tissue protector over the delivery pin 70 through the ilium, across the SI-Joint, and into the sacrum, until the proximal end of the threaded implant 10 is flush against the lateral wall of the ilium (see also FIGS. 8A-B). In various embodiments, the delivery pin 70 and soft tissue protector are withdrawn, leaving the threaded implant 10 residing in the bored passageway, flush with the lateral wall of the ilium (see FIGS. 8A-B). In other embodiments, the proximal end of the threaded implant 10 is left proud of the lateral wall of the ilium, such that it extends about 1 mm, 2 mm, 3 mm, 4 mm or 5 mm outside of the ilium. This ensures that the threaded implant 10 engages the hard cortical portion of the ilium rather than just the softer cancellous portion, through which it might migrate if there was no structural support from hard cortical bone. The hard cortical bone can also bear the loads or forces typically exerted on the bone by the threaded implant 10. In the illustrated embodiment, two additional threaded implants 10 are delivered in this manner, as FIG. 7 best shows.

The threaded implants 10 are sized according to the local anatomy. For the SI-Joint, representative threaded implants 10 may range in size, depending upon the local anatomy, from about 35 mm to 70 mm in length, and about a 7 mm 15 mm diameter. The morphology of the local structures can be generally understood by medical professionals using textbooks of human skeletal anatomy along with their knowledge of the site and its disease or injury. The physician is also able to ascertain the dimensions of the threaded implant 10 based upon prior analysis of the morphology of the targeted bone using, for example, plain film x-ray, fluoroscopic x-ray, or MRI or CT scanning, as well as intraoperative sizing methods using provided instrumentation.

The threaded implant structures can obviate the need for autologous grafts, bone graft material, additional screws and/or rods, hollow modular anchorage screws, cannulated compression screws, cages, or other fixation screws. Still, in the physician's discretion, bone graft material and other fixation instrumentation can be used in combination with the threaded implants.

The threaded implants make possible surgical techniques that are less invasive than traditional open surgery with no extensive soft tissue stripping. The assemblies make possible straightforward surgical approaches that complement the minimally invasive surgical techniques. The profile and design of the threaded implants minimize rotation and micro-motion. Rigid threaded implants made from titanium provide immediate post-op fusion stability. A bony in-growth, on-growth and/or through growth region including a porous plasma spray coating with irregular surface supports stable bone fixation/fusion. The threaded implants and surgical approaches make possible the placement of larger fusion surface areas designed to maximize post-surgical weight bearing capacity and provide a biomechanically rigorous implant designed specifically to stabilize the heavily loaded lumbar spine.

Additional details pertinent to the present invention, including materials and manufacturing techniques, may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the examples described herein, but only by the plain meaning of the claim terms employed.

What is claimed is:

1. A threaded bone implant comprising:
a body having a distal end and a proximal end, the body having a wall with an exterior surface, an interior surface, and a thickness through the wall between the surfaces;
a plurality of pores on the body, wherein each pore of the plurality of pores does not extend through the entire thickness of the wall between the interior and exterior surfaces; and
a slot located at the proximal end of the body, the threaded implant configured to be inserted laterally into sacral-iliac joint between an iliac and a sacrum.

2. The implant of claim 1, wherein the body is formed from a trabecular porous metal.

3. The implant of claim 1, wherein the body is formed from a solid metal coated with porous metal.

4. The implant of claim 1, wherein each pore of the plurality of pores does not directly connect the exterior and interior surfaces.

5. The implant of claim 1, wherein the plurality of pores have a mean pore size between about 50 to about 800 microns.

6. The implant of claim 1, wherein the body comprises a plurality of threads on an exterior surface of the body.

7. The implant of claim 6, wherein the threads comprise proximally directed barbs adapted to facilitate driving the implant into bone and to inhibit migration out of bone.

8. The implant of claim 1, wherein the threaded implant is formed from a material conducive to bony in-growth, on-growth or through-growth.

9. The implant of claim 1, further comprising a plurality of fenestrations adapted to promote bony in-growth.

10. The implant of claim 1, further comprising a surface coating adapted to promote bony in-growth.

11. The implant of claim 1, wherein the body comprises a first diameter at the distal end and a second diameter at the proximal end, the second diameter being greater in dimension that the first diameter.

12. The implant of claim 11, wherein the first and second diameters are between about 5 mm and 15 mm.

13. The implant of claim 1, wherein the body is tapered along a longitudinal direction between the distal and proximal ends.

14. A threaded bone implant comprising:
a hollow body having a distal end and a proximal end, the hollow body having a wall covered with a plurality of pores, wherein each pore of the plurality of pores has a mean pore size between about 50 microns to about 800 microns; and
a head located at the proximal end of the body, the threaded implant configured to be inserted into adjacent bone segments.

15. The implant of claim 14, wherein each pore of the plurality of pores does not penetrate through the entire thickness of the wall.

16. The implant of claim 14, wherein the body comprises a threaded portion.

17. The implant of claim 16, wherein the threaded portion comprises a pitch configured to stabilize the adjacent bone segments.

18. The implant of claim 17, wherein the pitch is between 0.5 mm to about 4 mm.

19. The implant of claim 16, wherein the body is formed from a trabecular porous metal.

20. A method for relieving joint pain comprising:
identifying a joint comprising a first bone segment, a second bone segment, and a non-bony region between the first and second bone segments;
providing a threaded bone fusion implant comprising a body with a threaded outer surface;
inserting a delivery pin through a first bone segment, through the non-bony region, and into a second bone segment;
forming a pilot bore in the first and second bone segments and through the non-bony region; and
inserting the threaded implant into the first and second bone segments to thereby fuse the bone segments.

21. The method of claim 20, wherein the implant body comprises a wall covered with a plurality of pores, wherein the plurality of pores has a mean pore size between about 50 microns to about 800 microns.

22. The method of claim 20, wherein the implant body comprises a wall with an exterior surface, an interior surface, and a thickness through the wall between the surfaces, the body including a plurality of pores and each pore of the plurality of pores does not extend through the entire thickness of the wall between the interior and exterior surfaces.

23. The method of claim 20, wherein inserting the threaded implant further comprises screwing the implant into the pilot bore.

24. The method of claim 20, wherein inserting the threaded implant further comprises tapping the implant into the pilot bore and positioning the implant in the first segment, through the non-bony region, and partially through the second bone segment.

25. The method of claim 20, wherein forming the pilot bore comprises shaping the pilot bore to have a tapered shape corresponding to a tapered section of the implant body.

26. A method for fusion of the sacral-iliac joint between an ilium and a sacrum, comprising:
inserting a threaded implant laterally near the ilium, the implant including a proximal end and a distal end; and
driving the distal end of threaded implant into the ilium, through the sacral-iliac joint, and into the sacrum.

27. The method of claim 26, wherein the implant comprises a body having a wall covered with a plurality of pores, wherein the plurality of pores has a mean pore size between about 50 microns to about 800 microns.

28. The method of claim 27, wherein the body comprises a wall with an exterior surface, an interior surface, and a thickness through the wall between the surfaces and each pore of the plurality of pores does not extend through the entire thickness of the wall between the interior and exterior surfaces.

29. The method of claim 26, wherein inserting the threaded implant further comprises screwing the implant through the ilium, through the sacral-iliac joint, and partially through the sacrum.

30. The method of claim 26, further comprising positioning the threaded implant by directly tapping the implant into through the sacral-iliac joint without forming a pilot bore.
